(19) United States
(12) Patent Application Publication
(10) Pub. No.: US 2023/0386113 A1
(43) Pub. Date: Nov. 30, 2023

(54) MEDICAL IMAGE PROCESSING APPARATUS AND MEDICAL IMAGE PROCESSING METHOD

(71) Applicants: Rigshospitalet, Copenhagen (DK); CANON MEDICAL SYSTEMS CORPORATION, Tochigi (JP)

(72) Inventors: Takahiko NISHIOKA, Otawara-shi (JP); Klaus FUGLSANG KOFOED, Copenhagen (DK); Mathias BECH MOELLER, Copenhagen (DK)

(73) Assignees: Rigshospitalet, Copenhagen (DK); CANON MEDICAL SYSTEMS CORPORATION, Tochigi (JP)

(21) Appl. No.: 18/322,719

(22) Filed: May 24, 2023

(30) Foreign Application Priority Data
May 26, 2022 (JP) 2022-085830

(51) Int. Cl. G06T 11/60; G06T 7/00; G06V 20/50; G06T 7/70; G16H 30/40

(52) U.S. Cl. CPC G06T 11/60; G06T 7/0012; G06V 20/50; G06T 7/70; G16H 30/40; G06T 2207/30048; G06T 2207/30101; G06V 2201/031; G06T 2210/41

(57) ABSTRACT

A medical image processing apparatus according to an embodiment includes processing circuitry. The processing circuitry is configured to obtain a medical image related to the heart. The processing circuitry is configured to generate a polar map indicating myocardial function information on the basis of the medical image. The processing circuitry is configured to cause a display to display a blood vessel image indicating forms of blood vessels included in the heart so as to be superimposed on the polar map. The processing circuitry is configured to receive an operation to designate at least one of the blood vessels displayed over the polar map. The processing circuitry is configured to identify information associated with the blood vessel designated by the operation. The processing circuitry is configured to cause the display to display the information associated with the blood vessel.

### CROSS-REFERENCE TO RELATED APPLICATIONS

This application is based upon and claims the benefit of priority from Japanese Patent Application No. 2022-085830, filed on May 26, 2022; the entire contents of which are incorporated herein by reference.

### FIELD

Embodiments described herein relate generally to a medical image processing apparatus and a medical image processing method.

### BACKGROUND

Conventionally, a technique is known by which a polar map indicating myocardial function information is generated on the basis of a medical image related to the heart, so as to assist a myocardial analysis by displaying a blood vessel image indicating forms of blood vessels included in the heart so as to be superimposed on the polar map.

However, because the heart includes a plurality of types of blood vessels, it may be difficult in some situations to understand the types of the blood vessels displayed over the polar map, when the blood vessel image indicating the blood vessels is simply displayed.

### BRIEF DESCRIPTION OF THE DRAWINGS

FIG. 1 is a diagram illustrating an exemplary configuration of a medical image processing apparatus according to a first embodiment;

FIG. 2 is a drawing illustrating an example of a polar map generated by a generating function according to the first embodiment;

FIG. 3 is a drawing illustrating examples of the polar map and a blood vessel image displayed by a first display controlling function according to the first embodiment;

FIG. 4 is a drawing illustrating an example of an information display realized by a second display controlling function according to the first embodiment;

FIG. 5 is a drawing illustrating another example of the information display realized by the second display controlling function according to the first embodiment;

FIG. 6 is a flowchart illustrating a processing procedure in a process performed by processing circuitry of the medical image processing apparatus according to the first embodiment;

FIG. 7 is a drawing illustrating an example of an information display realized by a second display controlling function according to a second embodiment; and

FIG. 8 is a drawing illustrating an example of an information display realized by a second display controlling function according to a third embodiment.

### DETAILED DESCRIPTION

A medical image processing apparatus according to an embodiment includes an obtaining unit, a generating unit, a first display controlling unit, a receiving unit, an identifying unit, and a second display controlling unit. The obtaining unit is configured to obtain a medical image related to the heart. The generating unit is configured to generate a polar map indicating myocardial function information on the basis of the medical image. The first display controlling unit is configured to cause a display to display a blood vessel image indicating forms of blood vessels included in the heart so as to be superimposed on the polar map. The receiving unit is configured to receive an operation to designate at least one of the blood vessels displayed over the polar map. The identifying unit is configured to identify information associated with the blood vessel designated by the operation. The second display controlling unit is configured to cause the display to display the information associated with the blood vessel.

Exemplary embodiments of a medical image processing apparatus and a medical image processing method will be explained in detail below, with reference to the accompanying drawings.

### First Embodiment

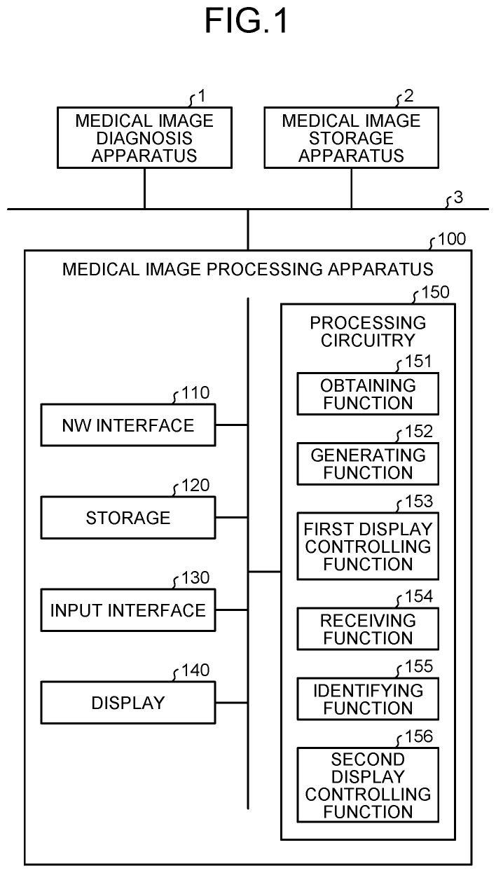

FIG. 1 is a diagram illustrating an exemplary configuration of a medical image processing apparatus according to a first embodiment.

For example, as illustrated in FIG. 1, a medical image processing apparatus 100 according to the present embodiment is connected, via a network 3, to a medical image diagnosis apparatus 1 and to a medical image storage apparatus 2, so as to be able to communicate with each other. In addition, the medical image processing apparatus 100 may further be connected to one or more other apparatuses (not illustrated) via the network 3.

The medical image diagnosis apparatus 1 is configured to acquire a medical image related to a subject. For example, the medical image diagnosis apparatus 1 may be an X-ray Computed Tomography (CT) apparatus, a Magnetic Resonance Imaging (MRI) apparatus, an ultrasound diagnosis apparatus, an X-ray diagnosis apparatus, a Positron Emission Tomography (PET) apparatus, a Single Photon Emission Computed Tomography (SPECT) apparatus, a PET-CT apparatus in which a PET apparatus and an X-ray CT apparatus are integrally formed, a SPECT-CT apparatus in which a SPECT apparatus an and X-ray CT apparatus are integrally formed, or the like.

The medical image storage apparatus 2 is configured to store therein the medical image acquired by the medical image diagnosis apparatus 1. For example, the medical image storage apparatus 2 is realized by using a computer machine such as a Picture Archiving and Communication System (PACS) server and is configured to store the medical image therein in a format compliant with a Digital Imaging and Communications in Medicine (DICOM) scheme.

The medical image processing apparatus 100 is configured to process the medical image related to the subject. More specifically, the medical image processing apparatus 100 is configured to obtain the medical image from either the medical image diagnosis apparatus 1 or the medical image storage apparatus 2 via the network 3 and to process the obtained medical image. For example, the medical image processing apparatus 100 is realized by using a computer machine such as a workstation.

For example, the medical image processing apparatus 100 includes a network (NW) interface 110, storage 120, an input interface 130, a display 140, and processing circuitry 150.

The NW interface 110 is configured to control transfer of various types of data and communication performed between the medical image processing apparatus 100 and other apparatuses via the network 3. More specifically, the NW interface 110 is connected to the processing circuitry 150 and is configured to transmit data received from the other apparatuses to the processing circuitry 150 and to transmit data received from the processing circuitry 150 to any of the other apparatuses. For example, the NW interface 110 is realized by using a network card, a network adaptor, a Network Interface Controller (NIC), or the like.

The storage 120 is configured to store therein various types of data and various types of programs. More specifically, the storage 120 is connected to the processing circuitry 150 and is configured to store therein data received from the processing circuitry 150 and to read and transmit any of the data stored therein to the processing circuitry 150. For example, the storage 120 is realized by using a semiconductor memory element such as a Random Access Memory (RAM) or a flash memory, or a hard disk, an optical disk, or the like.

The input interface 130 is configured to receive operations to input various types of instructions and various types of information, from an operator. More specifically, the input interface 130 is connected to the processing circuitry 150 and is configured to convert the input operations received from the operator into electrical signals and to transmit the electrical signals to the processing circuitry 150. For example, the input interface 130 is realized by using a trackball, a switch button, a mouse, a keyboard, a touch-pad on which an input operation can be performed by touching an operation surface thereof, a touch-screen in which a display screen and a touch-pad are integrally formed, a contactless input interface using an optical sensor, an audio input interface, and/or the like. In the present disclosure, the input interface 130 does not necessarily have to include physical operation component parts such as a mouse, a keyboard, and/or the like. For instance, possible examples of the input interface 130 include electrical signal processing circuitry configured to receive an electrical signal corresponding to an input operation from an external input mechanism provided separately from the apparatus and to transmit the electrical signal to controlling circuitry.

The display 140 is configured to display various types of information and various types of data. More specifically, the display 140 is connected to the processing circuitry 150 and is configured to display the various types of information and the various types of data received from the processing circuitry 150. For example, the display 140 is realized by using a liquid crystal monitor, a Cathode Ray Tube (CRT) monitor, a touch panel, or the like.

The processing circuitry 150 is configured to perform various types of processes by controlling constituent elements included in the medical image processing apparatus 100. For example, the processing circuitry 150 is configured to perform the various types of processes in accordance with input operations received from the operator via the input interface 130. Further, for example, the processing circuitry 150 is configured to store the data received by the NW interface 110 from the other apparatuses into the storage 120. Also, for example, the processing circuitry 150 is configured to transmit data read from the storage 120 to any of the other apparatuses, by transmitting the data to the NW interface 110. Further, for example, the processing circuitry 150 is configured to cause the display 140 to display any of the data read from the storage 120.

A configuration of the medical image processing apparatus 100 according to the present embodiment has thus been explained. The medical image processing apparatus 100 structured as described above has a function of assisting myocardial analyses, by generating a polar map indicating myocardial function information on the basis of a medical image related to the heart and displaying a blood vessel image indicating forms of blood vessels included in the heart so as to be superimposed on the polar map.

In this regard, because the heart includes a plurality of types of blood vessels, it may be difficult in some situations to understand the types of the blood vessels displayed over the polar map, when the blood vessel image indicating the blood vessels is simply displayed.

To cope with those situations, the medical image processing apparatus 100 according to the present embodiment is configured to make it possible to easily understand the types of the blood vessels displayed over the polar map. More specifically, the medical image processing apparatus 100 is configured to receive an operation to designate at least one of the blood vessels displayed over the polar map and to identify and display information associated with the blood vessel designated by the operation. In the present embodiment, an example will be explained in which the information associated with the blood vessel is a blood vessel name of the blood vessel.

Next, this configuration of the medical image processing apparatus 100 will be explained in detail.

For example, as illustrated in FIG. 1, the medical image processing apparatus 100 has, as processing functions included in the processing circuitry 150, an obtaining function 151, a generating function 152, a first display controlling function 153, a receiving function 154, an identifying function 155, and a second display controlling function 156.

In this situation, the obtaining function 151 is an example of the obtaining unit. The generating function 152 is an example of the generating unit. The first display controlling function 153 is an example of the first display controlling unit. The receiving function 154 is an example of the receiving unit. The identifying function 155 is an example of the identifying unit. The second display controlling function 156 is an example of the second display controlling unit.

The obtaining function 151 is configured to obtain a medical image related to the heart.

More specifically, the obtaining function 151 is configured to obtain the medical image related to the heart of a subject undergoing a medical examination, from either the medical image diagnosis apparatus 1 or the medical image storage apparatus 2 via the NW interface 110 and the network 3. After that, the obtaining function 151 is configured to store the obtained medical image into the storage 120.

For example, the obtaining function 151 is configured to receive, from the operator, subject identification information identifying the subject undergoing the medical examination, via the input interface 130. After that, the obtaining function 151 is configured to obtain the medical image related to the heart of the subject undergoing the medical examination, on the basis of the received subject identification information.

For example, the obtaining function 151 is configured to obtain a three-dimensional image (volume data) corresponding to multiple temporal phases related to the heart.

In this situation, the medical image obtained by the obtaining function 151 may be an image of any type, as long as it is possible to obtain the myocardial function information from the image. For example, the medical image may be a myocardial contrast-enhanced CT image obtained as a result of an X-ray CT apparatus imaging the heart of the subject for whom a contrast agent is administered. In another example, the medical image may be a myocardial Magnetic Resonance (MR) image obtained as a result of an MRI apparatus imaging the heart of the subject, while implementing an imaging method such as Arterial Spin Labeling (ASL) by which it is possible to capture a hemodynamics image without using a contrast agent.

The generating function 152 is configured to generate the polar map indicating the myocardial function information, on the basis of the medical image obtained by the obtaining function 151.

More specifically, the generating function 152 is configured to read, from the storage 120, the medical image related to the heart of the subject obtained by the obtaining function 151 and to generate the polar map indicating the myocardial function information on the basis of the read medical image.

For example, the generating function 152 is configured to generate the polar map indicating the myocardial function information by analyzing the three-dimensional image corresponding to the multiple temporal phases related to the heart that was acquired by the obtaining function 151.

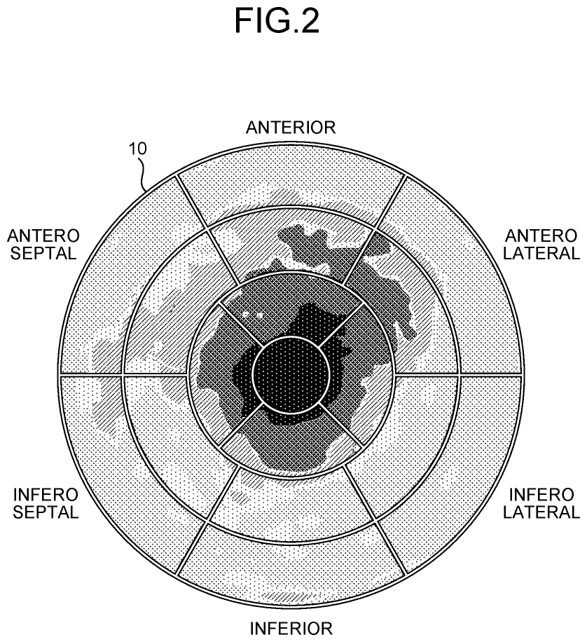

FIG. 2 is a drawing illustrating an example of the polar map generated by the generating function 152 according to the first embodiment.

For example, as illustrated in FIG. 2, a polar map 10 is an image in which, while a three-dimensional shape of myocardia is expanded on a plane so as to be simulatively expressed with a circular shape, the myocardial functional information is mapped onto the shape. For example, the polar map 10 illustrated in FIG. 2 indicates a myocardia region divided in six regions in the circumferential direction of the circle. “Anterior” denotes a front wall region; “Antero Lateral” denotes the front lateral wall; “Infero Lateral” denotes the lower lateral wall; “Inferior” denotes the lower wall; “Infero Septal” denotes the lower wall septum; and “Antero Septal” denotes the front wall septum.

More specifically, in the polar map 10, function information corresponding to different positions in the myocardia is mapped on the basis of a polar coordinate system defined by angles around a cardiac axis and distances from a cardiac apex part or a cardiac base part. In this situation, as the myocardial function information mapped on the polar map, it is possible to use any of various types of function indices. For example, the function information may be an arrival time of the contrast agent. In another example, the function information may be a myocardial wall thickness change rate, a volume change rate, a myocardial blood flow evaluation, or a myocardial viability evaluation. These types of function information are displayed over the polar map in colors assigned in advance in correspondence with values of the function indices.

The first display controlling function 153 is configured to cause the display 140 to display the blood vessel image indicating the forms of the blood vessels included in the heart so as to be superimposed on the polar map generated by the generating function 152.

More specifically, the first display controlling function 153 is configured to generate the blood vessel image indicating the forms of the blood vessels, by reading, from the storage 120, the medical image related to the heart of the subject obtained by the obtaining function 151 and extracting regions of the blood vessels from the read medical image. Further, the first display controlling function 153 is configured to align the position of the generated blood vessel image with the polar map generated by the generating function 152, so as to be displayed on the display 140 while being superimposed on the polar map.

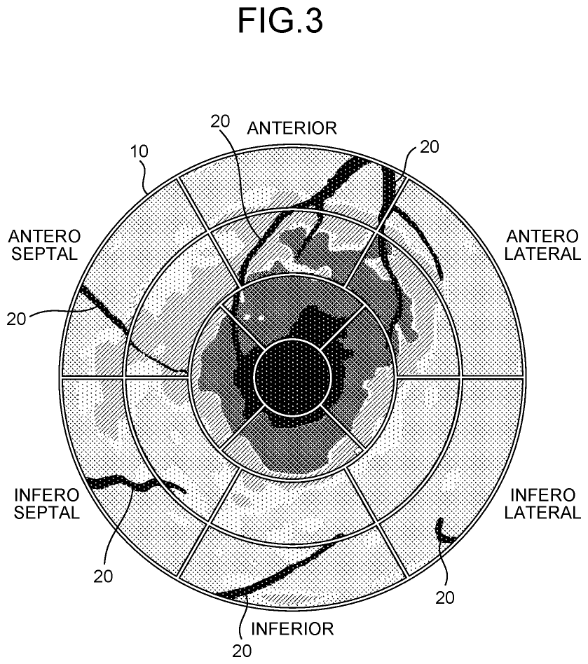

FIG. 3 is a drawing illustrating examples of the polar map and the blood vessel image displayed by the first display controlling function 153 according to the first embodiment.

For example, as illustrated in FIG. 3, with respect to a plurality of blood vessels included in the heart, the first display controlling function 153 is configured to display a blood vessel image 20 indicating the blood vessels so as to be superimposed on the polar map 10.

In this situation, for example, the first display controlling function 153 may generate the blood vessel image by using a medical image different from the medical image used for generating the polar map. For example, the first display controlling function 153 may generate the blood vessel image by using an X-ray fluoroscopic image of the heart of the same subject taken by an X-ray diagnosis apparatus or a Magnetic Resonance Angiography (MRA) image of the heart of the same subject imaged by an MRI apparatus. In those situations, the medical image used for generating the blood vessel image is, for example, obtained by the obtaining function 151 described above, from either the medical image diagnosis apparatus 1 or the medical image storage apparatus 2, together with the medical image used for generating the polar map.

In another example, instead of using the medical image of the subject, the first display controlling function 153 may generate the blood vessel image by using a model image indicating standard forms of the blood vessels included in the heart. In that situation, the first display controlling function 153 may use the model image as the blood vessel image without any modification or may generate the blood vessel image by complementing the blood vessel region extracted from the medical image with the model image. For example, the first display controlling function 153 may generate the blood vessel image by complementing the blood vessel region with the model image, when the blood vessel region generated from the medical image is partially disrupted or when the length of the blood vessel region does not satisfy a threshold value determined in advance for each blood vessel.

In yet another example, the first display controlling function 153 may generate the blood vessel image of the blood vessels included in the heart, by using a trained model generated through machine learning such as deep learning. For example, the first display controlling function 153 may generate the blood vessel image by using the trained model configured, upon receipt of an input of a heart medical image, to estimate and output a blood vessel image indicating standard forms of the blood vessels of the heart included in the medical image. In that situation, the trained model is generated in advance through machine learning using training data and stored in the storage 120.

The receiving function 154 is configured to receive an operation to designate at least one of the blood vessels displayed over the polar map.

More specifically, after the first display controlling function 153 displays the polar map and the blood vessel image, the receiving function 154 is configured to receive, from the operator, the operation to designate at least one of the blood vessels displayed over the polar map, via the input interface 130.

For example, the receiving function 154 is configured to receive, from the operator, the operation to select at least one of the plurality of blood vessels displayed over the polar map.

For example, the receiving function 154 may receive an operation (which may be called “mouse hover” or “mouseover”) to position a mouse pointer over the blood vessel image of the one of the blood vessels displayed over the polar map, as the operation to designate the one of the blood vessels. In another example, the receiving function 154 may receive an operation to click with a mouse on each of the blood vessel images of the plurality of blood vessel displayed over the polar map, as an operation to designate the plurality of blood vessels.

The identifying function 155 is configured to identify a blood vessel name of the blood vessel designated by the operation received by the receiving function 154.

More specifically, when the receiving function 154 has received the operation to designate at least one of the blood vessels displayed over the polar map, the identifying function 155 is configured to identify the blood vessel name of the blood vessel designated by the operation.

For example, with respect to the designated blood vessel, the identifying function 155 is configured to determine the position of the blood vessel in the heart, on the basis of the medical image related to the heart of the subject. Further, the identifying function 155 is configured to identify the blood vessel name of the designated blood vessel, by comparing the determined position of the blood vessel with information keeping positions of a plurality of blood vessels included in the heart in correspondence with information associated with the plurality of blood vessels. In that situation, the information keeping the blood vessel positions in correspondence with the blood vessel names is generated in advance and stored in the storage 120.

Alternatively, for example, the identifying function 155 may identify the blood vessel name of the designated blood vessel, by using a trained model generated through machine learning such as deep learning. For example, the identifying function 155 may identify the blood vessel name, by using the trained model configured to receive an input of a blood vessel image of a blood vessel included in the heart and to estimate and output the blood vessel name of the blood vessel. In that situation, the trained model is generated in advance through machine learning using training data and stored in the storage 120.

The second display controlling function 156 is configured to cause the display 140 to display the blood vessel name identified by the identifying function 155.

More specifically, after the identifying function 155 identified the blood vessel name, the second display controlling function 156 is configured to cause the display 140 to display information indicating the blood vessel name together with the polar map and the blood vessel image.

For example, the second display controlling function 156 is configured to display the information indicating the blood vessel name, in the vicinity of the blood vessel image of the designated blood vessel displayed over the polar map.

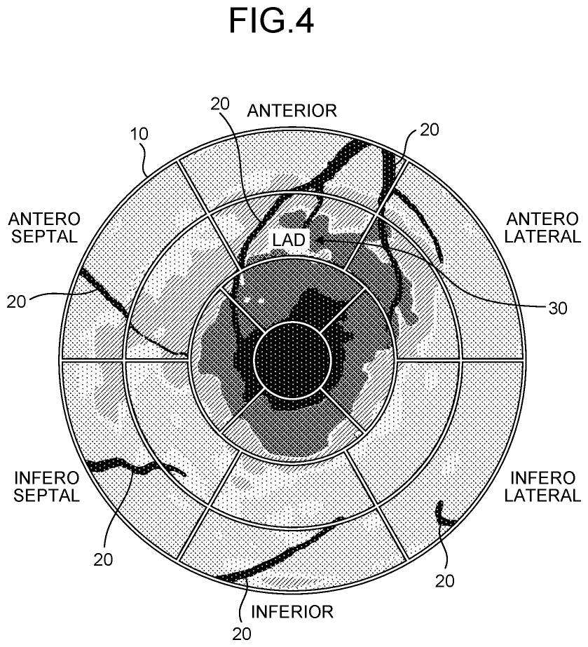

FIG. 4 is a drawing illustrating an example of an information display realized by the second display controlling function 156 according to the first embodiment.

For example, as illustrated in FIG. 4, when the left anterior descending artery is designated from among the plurality of blood vessels displayed over the polar map 10, the second display controlling function 156 is configured to display text 30 reading “LAD” (Left Anterior Descending artery) identifying the blood vessel name of the left anterior descending artery, in the vicinity of the blood vessel image 20 of the left anterior descending artery displayed over the polar map.

Alternatively, for example, the second display controlling function 156 may display the blood vessel name outside the polar map, so as not to obstruct observation of the myocardial function information displayed in the polar map.

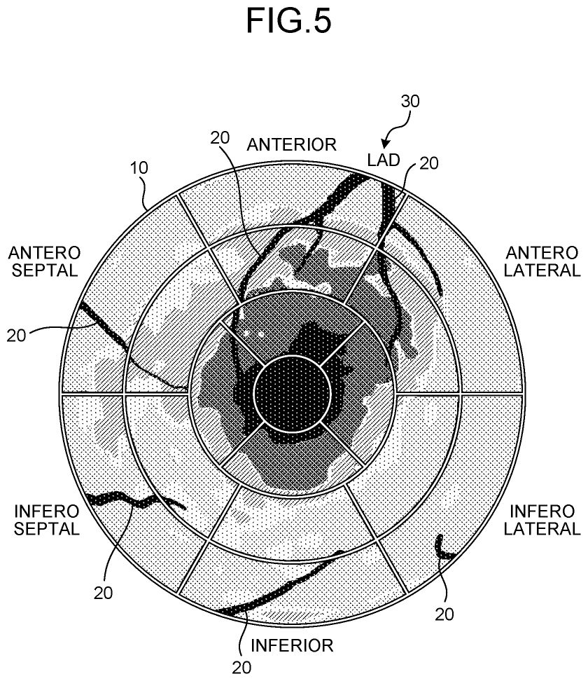

FIG. 5 is a drawing illustrating another example of the information display realized by the second display controlling function 156 according to the first embodiment.

For example, as illustrated in FIG. 5, when the left anterior descending artery is designated from among the plurality of blood vessels displayed over the polar map 10, the second display controlling function 156 may display the text 30 reading “LAD” (Left Anterior Descending artery) identifying the blood vessel name of the left anterior descending artery, in the vicinity of the blood vessel image 20 of the left anterior descending artery outside the polar map.

In these situations, for example, the second display controlling function 156 may be configured, while the mouse pointer is placed on the blood vessel image of the blood vessel displayed over the polar map, to keep displaying the blood vessel name and may be configured, when the mouse pointer is no longer placed on the blood vessel image, to bring the blood vessel name into a non-display state. Alternatively, for example, the second display controlling function 156 may be configured, when the blood vessel image of a blood vessel displayed over the polar map is clicked with a mouse, to display the blood vessel name of the blood vessel. In that situation, the second display controlling function 156 may display only the blood vessel name of the designated blood vessel as illustrated in the examples in FIGS. 3 and 4 or may display the blood vessel names of all the blood vessels including the other blood vessels at the same time.

The processing functions included in the processing circuitry 150 of the medical image processing apparatus 100 has thus been explained. In this situation, for example, the processing circuitry 150 is realized by using one or more processors. In that situation, for example, the processing functions described above are stored in the storage 120 in the form of computer-executable programs. Further, the processing circuitry 150 is configured to realize the functions corresponding to the programs, by reading and executing the programs stored in the storage 120. In other words, the processing circuitry 150 that has read the programs has the processing functions illustrated in FIG. 1.

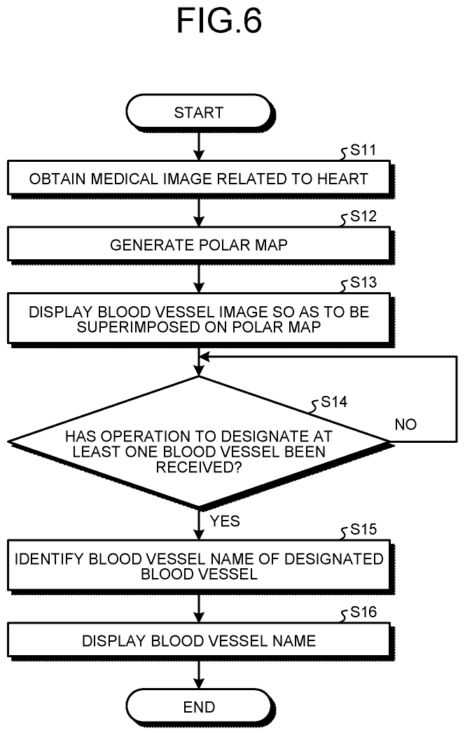

FIG. 6 is a flowchart illustrating a processing procedure in a process performed by processing circuitry 150 of the medical image processing apparatus 100 according to the first embodiment.

For example, as illustrated in FIG. 6, the processing circuitry 150 obtains the medical image related to the heart of the subject undergoing the medical examination, from either the medical image diagnosis apparatus 1 or the medical image storage apparatus 2 (step S11). This step is a step corresponding to the obtaining function 151 described above. For example, the processing circuitry 150 performs this step by reading and executing the program corresponding to the obtaining function 151 from the storage 120.

Subsequently, on the basis of the obtained medical image, the processing circuitry 150 generates the polar map indicating the myocardial function information (step S12). This step is a step corresponding to the generating function 152 described above. For example, the processing circuitry 150 performs this step by reading and executing the program corresponding to the generating function 152 from the storage 120.

After that, the processing circuitry 150 causes the display 140 to display the blood vessel image indicating the forms of the blood vessels included in the heart so as to be superimposed on the polar map (step S13). This step is a step corresponding to the first display controlling function 153 described above. For example, the processing circuitry 150 performs this step by reading and executing the program corresponding to the first display controlling function 153 from the storage 120.

Subsequently, upon receipt of an operation to designate at least one of the blood vessels displayed over the polar map (step S14: Yes), the processing circuitry 150 identifies the blood vessel name of the blood vessel designated by the operation (step S15). This step is a step corresponding to the receiving function 154 and the identifying function 155 described above. For example, the processing circuitry 150 performs this step by reading and executing the programs corresponding to the receiving function 154 and the identifying function 155 from the storage 120.

After that, the processing circuitry 150 causes the display 140 to display the identified blood vessel name (step S16). This step is a step corresponding to the second display controlling function 156 described above. For example, the processing circuitry 150 performs this step by reading and executing the program corresponding to the second display controlling function 156 from the storage 120.

As explained above, in the first embodiment, the obtaining function 151 is configured to obtain the medical image related to the heart. The generating function 152 is configured to generate the polar map indicating the myocardial function information on the basis of the obtained medical image. The first display controlling function 153 is configured to cause the display to display the blood vessel image indicating the forms of the blood vessels included in the heart so as to be superimposed on the polar map. The receiving function 154 is configured to receive the operation to designate at least one of the blood vessels displayed over the polar map. The identifying function 155 is configured to identify the blood vessel name of the blood vessel designated by the operation. The second display controlling function 156 is configured to cause the display to display the identified blood vessel name. Consequently, the first embodiment makes it possible to easily understand the types of the blood vessels displayed over the polar map.

The first embodiment has thus been explained. As for the medical image processing apparatus 100 described above, a part of the configuration thereof may be carried out as being modified as appropriate. Thus, in the following sections, modification examples related to the first embodiment will be explained as other embodiments. In the following embodiments, differences from the first embodiment will primarily be explained. Detailed explanations of certain aspects that are duplicate of what was explained above will be omitted.

### Second Embodiment

For example, in the first embodiment above, the medical image processing apparatus 100 is configured to display the blood vessel name of the designated blood vessel. In addition, it is also acceptable to further display information indicating whether or not treatment has been applied to the blood vessel. In the following sections, this example will be explained as a second embodiment.

In the present embodiment, the obtaining function 151 is configured to further obtain treatment history information of the subject undergoing the medical examination, from an electronic chart system, an image interpretation report generating apparatus, or the like, via the NW interface 110 and the network 3.

For example, the obtaining function 151 is configured to obtain the treatment history information of the subject undergoing the medical examination, on the basis of the subject identification information received from the operator.

After that, the identifying function 155 is configured to further identify whether or not treatment has been applied to the blood vessel designated by the operation received by the receiving function 154.

More specifically, the identifying function 155 is configured to identify whether or not treatment has been applied to the designated blood vessel, on the basis of the treatment history information obtained by the obtaining function 151. In this situation, examples of the treatment applied to the blood vessel include stent treatment and bypass treatment.

Further, together with the blood vessel name, the second display controlling function 156 is configured to cause the display 140 to further display information indicates whether or not treatment has been applied which was identified by the identifying function 155.

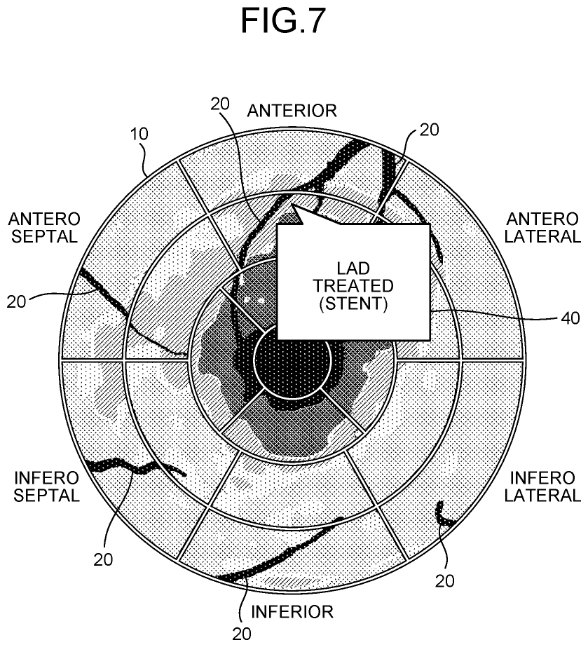

FIG. 7 is a drawing illustrating an example of the information display realized by the second display controlling function 156 according to the second embodiment.

For example, as illustrated in FIG. 7, when the left anterior descending artery is designated from among the plurality of blood vessel displayed over the polar map 10, the second display controlling function 156 is configured to display, in the vicinity of the blood vessel image 20 of the left anterior descending artery over the polar map, information 40 listing text “LAD” identifying the blood vessel name of the left anterior descending artery and text “TREATED (STENT)” indicating that stent treatment has been applied to the left anterior descending artery.

In this situation, for example, the second display controlling function 156 may be configured, while the mouse pointer is placed over the position within the blood vessel image to which the treatment has been applied, to display the information indicating that the positioned was already treated and may be configured, when the mouse pointer is placed over a position to which no treatment has been applied, to display information indicating that the position is untreated. In another example, the second display controlling function 156 may be configured, when the mouse pointer is placed over a position within the blood vessel image to which no treatment has been applied, to display information indicating whether the position is on the upstream side or the downstream side of the treatment location.

In yet another example, the second display controlling function 156 may be configured, when treatment using a treatment device such as a stent has been applied to the designated blood vessel, to display a range in which the treatment device is placed within the blood vessel image.

As explained above, in the second embodiment, the identifying function 155 is configured to further identify whether or not treatment has been applied to the designated blood vessel. Also, the second display controlling function 156 is configured to cause the display 140 to further display the identified information indicating whether or not treatment has been applied, together with the blood vessel name. Consequently, the second embodiment makes it possible to easily understand whether or not treatment has been applied to the blood vessel displayed over the polar map.

### Third Embodiment

For example, in the second embodiment, the medical image processing apparatus 100 is configured to display the blood vessel name of the designated blood vessel and the information indicating whether or not treatment has been applied to the blood vessel. It is also acceptable to further display the type of a disease occurring in the blood vessel and/or an image of the inside of the blood vessel (hereinafter, intravascular image). Next, this example will be explained as a third embodiment.

In the present embodiment, the obtaining function 151 is configured to further obtain disease information of the subject undergoing the medical examination, from an electronic chart system, an image interpretation report generating apparatus, or the like, via the NW interface 110 and the network 3. In addition, the obtaining function 151 is configured to further obtain the intravascular image related to the blood vessel designated by the operation received by the receiving function 154, from the medical image diagnosis apparatus 1, the medical image storage apparatus 2, or the like, via the NW interface 110 and the network 3.

For example, the obtaining function 151 is configured to obtain the disease information of the subject undergoing the medical examination and the intravascular image, on the basis of the subject identification information received from the operator.

In this situation, the intravascular image obtained by the obtaining function 151 may be any type of image as long as the image serves as a basis of treatment. For example, the intravascular image may be an Intravascular Ultrasound (IVUS) image, an Optical Coherence Tomography (OCT) image, a virtual endoscopy image (which may be called a fly-through image), or the like.

Further, the identifying function 155 is configured to further identify the type of a disease occurring in the blood vessel designated by the operation received by the receiving function 154.

More specifically, the identifying function 155 is configured to identify the type of the disease occurring in the designated blood vessel, on the basis of the disease information obtained by the obtaining function 151. In this situation, the type of the disease may be, for example, calcific (calcium) stenosis, plaque stenosis, malformation, or the like.

Further, the second display controlling function 156 is configured to cause the display 140 to further display information indicating the type of the disease identified by the identifying function 155, together with the blood vessel name. In addition, the second display controlling function 156 is configured to further display the intravascular image obtained by the obtaining function 151, together with the blood vessel name.

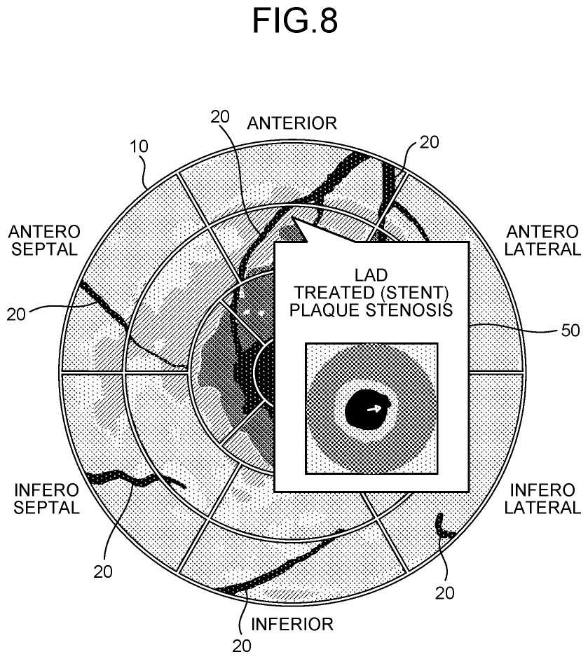

FIG. 8 is a drawing illustrating an example of the information display realized by the second display controlling function 156 according to the third embodiment.

For example, as illustrated in FIG. 8, when the left anterior descending artery is designated from among the plurality of blood vessels displayed over the polar map 10, the second display controlling function 156 is configured to display, in the vicinity of the blood vessel image 20 of the left anterior descending artery over the polar map, information 50 listing the text “LAD” identifying the blood vessel name of the left anterior descending artery, the text “TREATED (STENT)” indicating that stent treatment has been applied to the left anterior descending artery, the text “PLAQUE STENOSIS” indicating that the left anterior descending artery has plaque stenosis, and an image of the inside of the left anterior descending artery.

In this situation, for example, the second display controlling function 156 may display intravascular images before and after the treatment, as the intravascular image. In that situation, the obtaining function 151 is configured to obtain the intravascular images before and after the treatment related to the designated blood vessel, from the medical image diagnosis apparatus 1, the medical image storage apparatus 2, or the like. Further, the second display controlling function 156 is configured to display the intravascular images before and after the treatment obtained by the obtaining function 151, together with the blood vessel name.

As explained above, in the third embodiment, the identifying function 155 is configured to further identify the type of the disease occurring in the designated blood vessel. Also, the second display controlling function 156 is configured to further display the information indicating the type of the disease together with the blood vessel name. Consequently, the third embodiment makes it possible to easily understand the type of the disease occurring in the blood vessel displayed over the polar map.

Further, in the third embodiment, the obtaining function 151 is configured to further obtain the intravascular image related to the designated blood vessel. Further, the second display controlling function 156 is configured to further display the intravascular image together with the blood vessel name. Consequently, the third embodiment makes it possible to easily understand the state of the disease occurring in the blood vessel displayed over the polar map.

Further, in the third embodiment, the obtaining function 151 is configured to obtain the intravascular images before and after the treatment. Further, the second display controlling function 156 is configured to display the intravascular images before and after the treatment, together with the blood vessel name. Consequently, the third embodiment makes it possible to easily understand changes in the state of the disease occurring in the blood vessel displayed over the polar map.

In the third embodiment described above, the second display controlling function 156 is configured to display the information indicating whether or not treatment has been applied, the type of the disease, and the intravascular image, with respect to the designated blood vessel. However, these pieces of information do not all necessarily have to be displayed. For example, the second display controlling function 156 may be configured, in accordance with an instruction from the operator or a predetermined specification, to display, together with the blood vessel name, one or more pieces of information selected from among: the information indicating whether or not treatment has been applied, the type of the disease, and the intravascular image.

### Other Embodiments

It is possible to apply any of the configurations of the medical image processing apparatuses described in the above embodiments, to a system intermediated by a network such as a cloud. In that situation, for example, functions that are the same as or similar to the obtaining function, the generating function, and the identifying function described above are installed in processing circuitry of a server apparatus included in the system. In addition, for example, the first display controlling function, the receiving function, and the second display controlling function described above are installed in processing circuitry of a client apparatus used by a user of the system.

Further, it is also possible to apply any of the configurations of the medical image processing apparatuses described in the above embodiments, to a console apparatus of a medical image diagnosis apparatus or a medical image storage apparatus. In that situation, for example, functions that are the same as or similar to the obtaining function, the generating function, the first display controlling function, the receiving function, the identifying function, and the second display controlling function described above are installed in processing circuitry of the console apparatus of the medical image diagnosis apparatus or the medical image storage apparatus.

Furthermore, in the embodiments described above, the processing circuitry does not necessarily have to be realized by using a single processor and may be structured by combining together a plurality of independent processors, so that the processing functions are realized as a result of the processors executing the programs. Further, the processing functions of the processing circuitry may be realized as being distributed among or integrated into one or more pieces of processing circuitry as appropriate. Also, the processing functions of the processing circuitry may be realized by using a combination of hardware such as circuitry and software. Further, although the examples were explained above in which the programs corresponding to the processing functions are stored in the single storage, possible embodiments are not limited to these examples. For instance, the programs corresponding to the processing functions may be stored as being distributed among a plurality of storages, while the processing circuitry is configured to read and execute the programs from the storages.

Further, in the embodiments described above, the examples were explained in which the obtaining unit, the generating unit, the first display controlling unit, the receiving unit, the identifying unit, and the second display controlling unit of the present disclosure are realized as the obtaining function, the generating function, the first display controlling function, the receiving function, the identifying function, and the second display controlling function of the processing circuitry, respectively. However, possible embodiments are not limited to these examples. For example, instead of being realized as the obtaining function, the generating function, the first display controlling function, the receiving function, the identifying function, and the second display controlling function described in the embodiments, the functions of the obtaining unit, the generating unit, the first display controlling unit, the receiving unit, the identifying unit, and the second display controlling unit of the present disclosure may be realized by using hardware alone, software alone, or a combination of hardware and software.

Furthermore, the term “processor” used in the explanations of the above embodiments denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). In this regard, instead of having the programs saved in the storage, it is also acceptable to directly incorporate the programs into circuitry of one or more processors. In that situation, the one or more processors realize the functions by reading and executing the programs incorporated in the circuitry thereof. Further, the processors of the present embodiments do not each necessarily have to be structured as a single piece of circuitry. It is also acceptable to structure one processor by combining together a plurality of pieces of independent circuitry so as to realize the functions thereof.

In this situation, the programs executed by the processors are provided as being incorporated, in advance, into a Read-Only Memory (ROM), storage, or the like. Alternatively, the programs may be provided as being recorded on a non-transitory computer-readable storage medium such as a Compact Disk Read-Only Memory (CD-ROM), a Flexible Disk (FD), a Compact Disk Recordable (CD-R), a Digital Versatile Disk (DVD), or the like, in a file in a format that is either installable or executable for these apparatuses. Further, the programs may be stored in a computer connected to a network such as the Internet, so as to be provided or distributed as being downloaded via the network. For example, the programs are structured with modules including the processing functions described above. In the actual hardware, as a result of a CPU reading and executing the programs from a storage medium such as a ROM, the modules are loaded into a main storage apparatus so as to be generated in the main storage apparatus.

In addition, the constituent elements of the apparatuses illustrated in the drawings in the above embodiments are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a CPU and a program analyzed and executed by the CPU or may be realized as hardware using wired logic.

Furthermore, with regard to the processes explained in the above embodiments, it is acceptable to manually perform all or a part of the processes described as being performed automatically. Conversely, by using a publicly-known method, it is also acceptable to automatically perform all or a part of the processes described as being performed manually. Further, unless noted otherwise, it is acceptable to arbitrarily modify any of the processing procedures, the controlling procedures, specific names, and various information including various types of data and parameters that are presented in the above text and the drawings.

Further, various types of data handled in the present disclosure are, typically, digital data.

According to at least one aspect of the embodiments described above, it is possible to easily understand the types of the blood vessels displayed over the polar map.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured:
to obtain a medical image related to a heart;
to generate a polar map indicating myocardial function information on a basis of the medical image;
to cause a display to display a blood vessel image indicating forms of blood vessels included in the heart so as to be superimposed on the polar map;
to receive an operation to designate at least one of the blood vessels displayed over the polar map;
to identify information associated with the blood vessel designated by the operation; and
to cause the display to display the information associated with the blood vessel.

2. The medical image processing apparatus according to claim 1, wherein
the processing circuitry is configured to further identify whether or not treatment has been applied to the designated blood vessel, and
the processing circuitry is configured to further display information indicating whether or not the treatment has been applied, together with the information associated with the blood vessel.

3. The medical image processing apparatus according to claim 1, wherein
the processing circuitry is configured to further identify a type of a disease occurring in the designated blood vessel, and
the processing circuitry is configured to further display information indicating the type of the disease, together with the information associated with the blood vessel.

4. The medical image processing apparatus according to claim 1, wherein
the processing circuitry is configured to further obtain an intravascular image related to the designated blood vessel, and
the processing circuitry is configured to further display the intravascular image together with the information associated with the blood vessel.

5. The medical image processing apparatus according to claim 4, wherein
the processing circuitry is configured to obtain intravascular images before and after treatment, and
the processing circuitry is configured to display the intravascular images before and after the treatment, together with the information associated with the blood vessel.

6. The medical image processing apparatus according to claim 1, wherein
the processing circuitry is configured to further determine a position of the designated blood vessel, and
the processing circuitry is configured to identify the blood vessel name of the designated blood vessel by comparing the determined position of the blood vessel with information keeping positions of a plurality of blood vessels included in the heart in correspondence with pieces of information associated with the plurality of blood vessels.

7. The medical image processing apparatus according to claim 1, wherein the information associated with the blood vessel is a blood vessel name of the blood vessel.

8. A medical image processing method comprising:
causing processing circuitry to obtain a medical image related to a heart;
causing the processing circuitry to generate a polar map indicating myocardial function information on a basis of the medical image;
causing the processing circuitry to display, on a display, a blood vessel image indicating forms of blood vessels included in the heart so as to be superimposed on the polar map;
causing the processing circuitry to receive an operation to designate at least one of the blood vessels displayed over the polar map;
causing the processing circuitry to identify information associated with the blood vessel designated by the operation; and
causing the processing circuitry to display, on the display, the information associated with the blood vessel.
